(19) United States
(12) Patent Application Publication
(10) Pub. No.: US 2008/0008698 A1
(43) Pub. Date: Jan. 10, 2008

(54) Non-Surgical Method for Preventing or Reducing the Rate of the Progression of Non-Proliferative Diabetic Retinopathy and the Treatment of Other Ocular Conditions

(76) Inventors: Stephen P. Bartels, Pittsford, NY (US); Gregory L. McIntire, Rochester, NY (US); Timothy L. Comstock, Rochester, NY (US); Brian Levy, Rochester, NY (US)

Correspondence Address: Denis A. Polyn;Bausch & Lomb Incorporated, One Bausch & Lomb Place, Rochester, NY, 14604, US

(21) Appl. No.: 11/626,394

(22) Filed: Jan. 24, 2007

Related U.S. Application Data
(62) Division of application No. 11/126,625, filed on May 11, 2005.

(51) Int. Cl. A61K 38/48; A61P 27/02

(52) U.S. Cl. 424/94.64

(57) ABSTRACT

A non-surgical method for preventing or reducing the rate of the progression of non-proliferative diabetic retinopathy to the proliferative form of diabetic retinopathy comprising intravitreally administering to a patient suffering from non-proliferative diabetic retinopathy an effective amount of serine proteinase enzyme sufficient to create, without surgery, a posterior vitreal detachment to prevent or reduce the progression of proliferative diabetic retinopathy is said patient. Also disclosed is a non-surgical method of treating ocular conditions such as retinal ischemia, retinal inflammation, retinal edema tractional retinal detachment, tractional retinopathy, vitreous hemorrhage and tractional maculopathy by intravitreally administering to a patient suffering from one or more of these conditions with an effective amount of a serine proteinase enzyme to reduce or treat that particular ocular condition. Plasmin, microplasmin and miniplasmin are preferred serine proteinase enzymes and plasmin is the most preferred.

### CROSS-REFERENCE

This application is a divisional patent application and claims the benefit of U.S. application Ser. No. 11/126,625 filed May 11, 2005, which is incorporated herein by reference.

### FIELD OF THE INVENTION

This invention relates to a non-surgical method for preventing or reducing the rate of the progression of non-proliferative diabetic retinopathy to the proliferative form of diabetic retinopathy and to a non-surgical method for treating other ocular conditions such as retinal ischemia, retinal inflammation, retinal edema, macular hole, tractional retinal detachment, tractional retinopathies, vitreous hemorrhage and tractional maculopathy by administering intravitreally to a patient an effective amount of serine proteinase enzyme sufficient to create a posterior vitreal detachment without surgery.

### BACKGROUND OF THE INVENTION

Serine proteinase enzymes, including plasmin, microplasmin and miniplasmin are old and known. U.S. Pat. Nos. 2,624,691 and 3,234,106 disclose methods of purifying plasmin from blood. U.S. Pat. No. 4,774,087 discloses microplasmin and microplasminogen produced by the action of plasmin/plasminogen at high pH.

U.S. Pat. No. 5,304,118 discloses a method for performing a vitrectomy on an eye by introducing plasmin into the vitreous humor in order to induce a posterior vitreous detachment and thereafter removing the vitreous and replacing it with a sterile saline solution.

U.S. Pat. No. 5,637,299 discloses the enhancement of thrombolytic therapy with deglycosylated forms of plasminogen. U.S. Pat. No. 5,722,428 discloses a method for producing a posterior vitreous detachment using an enzyme that specifically cleaves type IV collagen and fibronectin to promote a partial or complete posterior vitreous detachment. U.S. Pat. No. 6,355,243 discloses a method of thrombolytic therapy by the direct administration of active plasmin to the clot site via catheter. U.S. Pat. No. 6,585,972 discloses a process for crosslinking of collagen in the vitreous of the eye and inducing separation of the posterior hyaloid from the retina. U.S. Pat. No. 6,733,750 discloses a process for inducing posterior vitreous detachment for dissolving blood clots in the vitreous by introducing a composition including plasminogen and a plasminogen activator enzyme into the ocular cavity of the eye. The foregoing composition is reported to induce substantially complete posterior vitreous detachment from the retina without causing unmanageable or serious inflammation of the retina and to dissolve blood clots in the vitreous. U.S. Pat. No. 6,787,135 discloses introducing plasmin into the vitreous in an amount sufficient to induce posterior detachment of the vitreous, mechanically detaching the vitreous from the eye, introducing a replacement fluid into the eye and introducing plasmin into the eye in an amount sufficient to decrease the total metalloproteinase activity in the vitreous.

Published U.S. patent application 2002/0042652 discloses a process for inhibiting vascular proliferation by introducing a composition into the eye inducing posterior vitreous detachment. The combination includes a combination of plasminogen, a collagen crosslinking agent and at least one plasminogen activator. The composition is introduced in the vitreous in an amount effective to induce crosslinking of the vitreous and to induce substantially complete or partial posterior vitreous detachment from the retina without causing inflammation of the retina. Published U.S. patent application 2002/0139378 discloses a method for creating a separation of posterior cortical vitreous from a retinal of the eye. The method includes the step of introducing plasmin into the vitreous humor of the eye. The plasmin may be introduced either by injection or by through a sustained release device. Published U.S. patent application 2002/0192794 discloses a process for producing a reversibly inactivated acidified plasmin that may be used in the administration of a thrombolytic therapy. Published U.S. patent application 2003/0026798 discloses a method of thrombolysis that allows the use of a fibrolytic composition comprising reversibly inactivated acidified plasmin and the localized delivery of the plasmin to a vascular thrombotic occlusion. Published U.S. patent application 2003/0113313 discloses a process for inhibiting vascular proliferation by separately introducing components into the eye to generate plasmin in the eye in amounts to induce complete posterior vitreous detachment where the vitreoretinal interface is devoid of cortical vitreous remnants. The process administers a combination of lysine-plasminogen, at least one recombinant plasminogen activator and thermolysin and a gaseous adjuvant to form a cavity in the vitreous. Published U.S. patent application 2003/0147877 discloses a process for liquefying vitreous humor of the eye. The process includes the step of delivering plasmin into the vitreous of the eye and incubating the vitreous and the plasmin together for a period of time. Plasmin may be introduced through injection or sustained release device and may be used to treat a pathological condition of the eye such as diabetic retinopathy, macular hole, macular pucker, intraocular infection, foreign intraocular material and retinal detachment.

Published U.S. patent application 2003/0175263 ('263) discloses methods of modifying total matrix metalloproteinase (MMP) activity in the vitreous of the eye. Enzyme assisted vitrectomy procedures are also disclosed and comprise introducing plasmin into the vitreous in an amount sufficient to induce posterior detachment of the vitreous, mechanically detaching the vitreous from the eye, introducing a replacement fluid into the eye and introducing plasmin into the replacement fluid in the eye in an amount sufficient to decrease the total metalloproteinase activity in vitreous. Paragraph 0006 of published U.S. patent application '263 states that one unit of plasmin activity is measured by the hydrolysis of a chromogenic substrated S-2251, citing a Friberger publication, and, preferably, that the amount of plasmin used to inhibit MMP activity in the vitreous post vitrectomy is less than one unit. The abstract of '263 states that the invention provides methods of inhibiting the progress of various disease conditions, including proliferative diabetic retinopathy. Less than one unit of plasmin is used to inhibit the progress of proliferative diabetic retinopathy after higher concentrations have been used to create a PVD followed within a short time period (0.5 to 2 hrs) by surgical removal of the inner limiting membrane. Hence, this concentration of plasmin can not induce a posterior vitreal detachment (PVD) in this paradigm inasmuch as the PVD has already been completed via pharmacological and surgical intervention. Paragraph 0020 of '263 states that in their method of performing a vitrectomy described in U.S. Pat. No. 5,304,118, the amount of plasmin needed to effect the posterior detachment of the vitreous before surgical vitrectomy is between 1 and 3 units of plasmin. Surprisingly, the applicants have discovered that much smaller amounts of plasmin can create a PVD when injected into the vitreous and allowed to remain therein without subsequent surgery. Applicants use an amount equivalent to about 0.5 to about 1000 μg of plasmin injected into the vitreous, preferably about 1.0 to 500 μg of plasmin injected into the vitreous, more preferably about 10 to 400 μg of plasmin injected into the vitreous, and, most preferably, about 50 to 200 μg of plasmin injected into the vitreous to prevent or reduce the rate of the progression of non-proliferative diabetic retinopathy to the proliferative form of diabetic retinopathy by creating a PVD without surgery. 1 unit as per the '263 method is equal to 4.7 international units (different substrate). Thus, <1 “unit” would equate with less than 4.7 IU. Applicants' plasmin is 22.5 μg/IU hence applicants' range of 0.5 to 1000 μg is equivalent to 0.02 IU to (approx) 44.4 IU or in '263 units, 0.005 to 9.45 IU. Applicants prevent or reduce the rate of the progression of non-proliferative diabetic retinopathy by inducing a PVD, not by inactivating the MMPs present in the vitreous. According to U.S. Pat. No. 5,304,118 ('118), it requires between 1 and 3 units of plasmin to induce PVD. According to '263, less than 1 unit of plasmin injected into a replacement fluid in the eye to inhibit the progress of proliferative diabetic retinopathy post surgical vitrectomy. In the context of '263, applicants use less than 1 unit of plasmin to prevent or reduce the rate of the progression of non-proliferative diabetic retinopathy and do so by inducing a PVD without surgery.

Published U.S. patent application 2004/0081643 discloses a process for inhibiting vascular proliferation by introducing a composition into the eye for inducing posterior vitreous detachment. The composition includes at least two compounds selected from the group consisting among other things plasmin and thermolysin in amount sufficient to induce a substantially complete or partial posterior vitreous detachment from the retina without causing inflammation of the retina and dissolve blood clots in the vitreous.

### SUMMARY OF THE INVENTION

This invention provides a non-surgical method for preventing or reducing the rate of the progression of non-proliferative diabetic retinopathy to the proliferative form of diabetic retinopathy by intravitreally administering to a patient suffering from non-proliferative diabetic retinopathy an effective amount of serine proteinase enzyme sufficient to create a posterior vitreal detachment without surgery. Preferably, the serine proteinase enzyme is selected from plasmin, microplasmin and miniplasmin. More preferably the serine proteinase enzyme is plasmin and the plasmin is obtained from plasminogen fractionated from human blood. The serine proteinase enzyme is administered intravitreally in an amount equivalent to about 0.5 to about 1000 μg of plasmin injected into the vitreous, preferably about 1.0 to 500 μg of plasmin injected into the vitreous, more preferably about 10 to 400 μg of plasmin injected into the vitreous, and, most preferably, about 50 to 200 μg of plasmin injected into the vitreous. This method is practiced without removal of the vitreous (e.g., vitrectomy) nor does it require inactivation of MMPs.

This invention also provides a non-surgical method for treating retinal ischemia, retinal inflammation, retinal edema, macular hole, tractional retinal detachment, tractional retinopathies, vitreous hemorrhage and tractional maculopathy by intravitreally administering to a patient suffering from one or more of those ocular conditions an effective amount of serine proteinase enzyme to reduce the retinal ischemia, retinal inflammation, retinal edema, macular hole, tractional retinal detachment, tractional retinopathies, vitreous hemorrhage and tractional maculopathy. More preferably, the serine proteinase enzyme is plasmin, microplasmin and miniplasmin. Most preferably, the serine proteinase enzyme is plasmin obtained from plasminogen fractionated from human blood. The serine proteinase enzyme used in this method is used in the same concentration ranges and administered in the same way as in the case of diabetic retinopathy.

### DETAILED DESCRIPTION OF PREFERRED EMBODIMENTS

This invention is useful for non-surgically preventing or reducing the rate of the progression of non-proliferative diabetic retinopathy to the proliferative form of diabetic retinopathy by intravitreally administering to a patient suffering from non-proliferative diabetic retinopathy an effective amount of serine proteinase enzyme sufficient to create a posterior vitreal detachment without surgery.

The serine proteinase enzyme used in the present invention may be plasmin, microplasmin, or miniplasmin or any form of plasmin that might otherwise result in the release of active plasmin, microplasmin, or miniplasmin in the vitreous of the eye either administered alone or in combination with an activating agent other than tissue plasminogen activator (tpa). More preferably, the plasmin, microplasmin or miniplasmin should be derived from or be identical in structure and function to human plasmin, microplasmin or miniplasmin. Most preferably, the serine proteinase enzyme used in this invention should be human plasmin derived from either human blood or via expression of human plasmin within yeast, bacteria, or single celled plants or mammalian cells that have been genetically modified so as to produce human plasmin or plasminogen, the native inactive precursor. In a preferred method, the serine proteinase enzyme should be administered in amounts of about 0.5 to about 1000 μg of plasmin injected into the vitreous, preferably about 1.0 to 500 μg of plasmin injected into the vitreous, more preferably about 10 to 400 μg of plasmin injected into the vitreous, and, most preferably, about 50 to 200 μg of plasmin injected into the vitreous to create a PVD.

The method can be practiced by intravitreally administering the serine proteinase enzyme by injection, or through a cannula. A preferred form of intravitreal administration is injection at multiple locations within the vitreous cavity. A more preferred form of intravitreal administration is injection in close proximity to the target tissue. A most preferred form of intravitreal administration is injection into the mid-vitreous while the head of the patient faces upward.

In addition to the method for preventing or reducing the rate of the progression of non-proliferative diabetic retinopathy, the serine proteinase enzyme can also be used to non-surgically treat retinal ischemia, retinal inflammation, retinal edema, macular hole, tractional retinal detachment, tractional retinopathies, vitreous hemorrhage and tractional maculopathy, by intravitreally administering to a patient suffering from one or more of these ocular conditions an effective amount of serine proteinase enzyme to treat that particular ocular condition. As in the case of the method for preventing or reducing the rate of the progression of non-proliferative diabetic retinopathy, the serine proteinase enzyme, the concentration, the method of administration can vary in similar fashion for the treatment of these disease conditions.

The method can be practiced by intravitreally administering the serine proteinase enzyme by injection of a solution containing the enzyme, injection of a solution containing the enzyme and additional excipients for control of pH, injection of a solution containing the enzyme and additional excipients for control of osmolality, injection of a solution containing the enzyme and additional excipients for control of pH and ionic strength and osmolality, injection of a solution containing the enzyme and additional excipients which provide stability to the enzyme during changes in pH as taught by Jensen (U.S. Pat. No. 3,950,513), injection of a solution containing the enzyme and additional excipients which provide optimal lyophilization of the serine proteinase enzyme including appearance of the freeze dried cake, reconstitution time using water or a mixture of water and nonaqueous solvent or a nonaqueous solvent alone, and preservation of activity of the enzyme.

The method can be practiced by intravitreally injecting a spreading agent (i.e., Vitrase®, hylauronidase, etc.) 30 min to 2 hr before intravitreally administering the serine proteinase enzyme by injection of a solution containing the enzyme, injection of a solution containing the enzyme and additional excipients for control of pH, injection of a solution containing the enzyme and additional excipients for control of osmolality, injection of a solution containing the enzyme and additional excipients for control of pH and ionic strength and osmolality, injection of a solution containing the enzyme and additional excipients which provide stability to the enzyme during changes in pH as taught by Jensen (U.S. Pat. No. 3,950,513), injection of a solution containing the enzyme and additional excipients which provide optimal lyophilization of the serine proteinase enzyme including appearance of the freeze dried cake, reconstitution time using water or a mixture of water and nonaqueous solvent or a nonaqueous solvent alone, and preservation of activity of the enzyme.

The method can further be practiced by intravitreally administering the serine proteinase enzyme by injecting a micellar solution containing the enzyme of interest, injecting a micellar solution containing the enzyme of interest and excipients that control pH and ionic strength, injecting a micellar solution containing the enzyme of interest and excipients that stabilize the enzyme to pH changes as taught by Jensen (U.S. Pat. No. 3,950,513), injecting a micellar solution containing the enzyme of interest wherein the surfactant composition of the micelle preferably affords a positively charge micelle, more preferably yields a negatively charged micelle, and most preferably results in a non charged (i.e., neutral) micelle, injecting a micellar solution containing the enzyme of interest wherein the surfactant composition of the micelle is primarily monomeric surfactant molecules, injecting a micellar solution containing the enzyme of interest wherein the composition of the micelle is primarily a nonionic polymeric surfactant or surfactants (e.g., Tweens, Spans, Pluronics, Tetronics, Myj, Brij, and polyethylene glycol (PEG)).

The method can further be practiced by intravitreally administering the serine proteinase enzyme by injection in a suspension either containing the enzyme or of the enzyme alone wherein the enzyme can be the solid suspended particle or present in solution in the solution phase of the particle suspension, injection of a suspension either containing the enzyme or of the enzyme alone wherein the enzyme can be the solid suspended particle or present in solution in the solution phase of the particle suspension together with excipients to control the pH of the solution phase of the suspension, injection of a suspension either containing the enzyme or of the enzyme alone wherein the enzyme can be the solid suspended particle or present in solution in the solution phase of the particle suspension with excipients to control the osmolality of the solution phase of the suspension, injection of a suspension either containing the enzyme or of the enzyme alone wherein the enzyme can be the solid suspended particle or present in solution in the solution phase with excipients to control the pH and ionic strength and osmolality of the solution phase of the suspension, injection of a suspension either containing the enzyme or of the enzyme alone wherein the enzyme can be the solid suspended particle or present in solution in the solution phase and additional excipients which provide stability to the enzyme during changes in pH as taught by Jensen (U.S. Pat. No. 3,950,513), injection of a suspension either containing the enzyme or of the enzyme alone wherein the enzyme can be the solid suspended particle or present in solution in the solution phase of the particle suspension with excipients which provide optimal lyophilization of the serine proteinase enzyme including appearance of the freeze dried cake, reconstitution time using water or a mixture of water and nonaqueous solvent or a nonaqueous solvent alone, and preservation of activity of the enzyme. It is further understood that by suspensions, it is meant to describe a dispersion of solid particles within a continuous liquid phase. Also, it is understood that these dispersions require special additives to afford physical stability and particle size control to the suspension such as surfactants and polymers as are well known in the art. Processes for production of such suspensions are well known in the art and are described in various textbooks (i.e., Remington, Martindale), regulatory guidelines (i.e., USP, EP, JP), and the literature including patents and publications all of which are herein embodied in this disclosure.

The method can further be practiced by intravitreally administering the serine proteinase enzyme by injection of a liposome solution containing the active enzyme resulting from a frozen liposome solution containing the enzyme or a lyophilized liposome solution containing the enzyme, injection of a liposome containing the active enzyme resulting from a frozen liposome solution containing the enzyme or a lyophilized liposome solution containing the enzyme together with excipients to control the pH of the aqueous phase of the liposome solution wherein the pH of the internal aqueous phase may be different than that of the external continuous solution phase, injection of a liposome containing the active enzye resulting from a frozen liposome solution containing the enzyme or a lyophilized liposome solution containing the enzyme with excipients to control the osmolality of the liposome solution, injection of a liposome containing the active enzyme resulting from a frozen liposome solution containing the enzyme or a lyophilized liposome solution containing the enzyme with excipients which provide optimal lyophilization of the serine proteinase enzyme liposome solution including appearance of the freeze dried cake, reconstitution time using water or a mixture of water and nonaqueous solvent or a nonaqueous solvent alone, and preservation of activity of the enzyme, injection of a liposome containing the active enzyme resulting from a frozen liposome solution containing the enzyme or a lyophilized liposome solution containing the enzyme with excipients that stabilize the enzyme to changes in pH as taught by Jensen (U.S. Pat. No. 3,950,513), ionic strength, and osmolality, and injection of a liposome containing an inactive precursor to the active serine proteinase. It is understood that in each case, the enzyme may reside within the liposome, outside of the excluded volume of the liposome or both within and outside the liposome bilayer and further that the term “liposome” may represent unilamellar vesicles, multilamellar vesicles, chocleates, and “niosome” vesicles where niosomes are known in the art as a nonaqueous core stabilized by a monolayer of phospholipids rather than the traditional bilayer of phospholipids. It is further understood that the composition of the bilayer in the liposome solution is also claimed in the delivery of these serine proteinase enzyme to the vitreous. Further, it is understood that individual particle size of the liposome solutions may vary from less than 80 nm to greater than 1000 nm. Finally, it is also noted that the liposomes of this description can be positively charged, negatively charged or relatively neutral in surface charge. In the case of charged liposomes it is conceivable that the individual phospholipid moieties may well complex with and thereby stabilize the plasmin to shifts in pH, osmolality, and/or tonicity for injection into the vitreous.

The method can further be practiced by intravitreally administering the serine proteinase enzyme by injection of an oil in water emulsion containing the enzyme of interest, injection of an oil in water emulsion containing the enzyme of interest and excipients to control pH, ionic strength and osmolality, injection of an oil in water emulsion containing the enzyme of interest and excipients that stabilize the enzyme to changes in pH as taught by Jensen (U.S. Pat. No. 3,950,513) and ionic strength, injection of an oil in water emulsion containing the enzyme of interest wherein the oil phase stabilizes the enzyme of interest to changes in pH. It is understood that the enzyme would most likely be resident in the continuous aqueous phase of these oil in water emulsions. However, those skilled in the art will recognize that the enzyme may also be dosed in water in oil emulsions wherein it will be resident in the water pockets suspended in the continuous nonaqueous phase. In that instance, the presence of various excipients within the aqueous pockets or in the continuous nonaqueous phase are also disclosed herein.

The method can further be practiced by intravitreally administering the serine proteinase enzyme by insertion of a rapidly dissolving tablet into the vitreous containing the enzyme of interest, insertion of a rapidly dissolving tablet into the vitreous containing the enzyme of interest and excipients to provide properties important in the preparation of tablets including compressibility, lubricity, hardness, and density, insertion of a rapidly dissolving tablet into the vitreous containing the enzyme of interest and excipients that stabilize the enzyme to changes in pH and ionic strength, insertion of a rapidly dissolving tablet into the vitreous containing the enzyme of interest and excipients that control the release of the active enzyme for periods of minutes to hours. Such tablets are known in the art and comprise Mini Tablets with dimensions of 0.5 mm<diameter<4 mm and more preferably 1.0 mm<diameter<2 mm and most preferably 1.25 mm<diameter<1.75 mm with length determined by dose (concentration of enzyme) in the tablet mixture. Generally, length is <10 mm and more preferably <5 mm and most preferably <2 mm.

The method can further be practiced by intravitreally administering the serine proteinase enzyme as a powder, as a powder mixed with excipients to control pH and ionic strength, as a powder mixed with excipients and suspended in nonaqueous solvents (e.g., mineral oil, vitamin e, silicon oil, perfluorocarbon oils, vegetable oils, peanut oil, safflower oil, glycerin, as a powder mixed with excipients and granulated into particles for administration into the eye, as a powder mixed with excipients and granulated and sieved for administration into the eye via aerosol or suspended in solvents as given above.

Any of the above methods of practice can be preferred methods of practice. A more preferred method of practice is the injection of a clear solution into the eye and a most preferred method of practice is the injection of a clear solution into the eye containing excipients which stabilize the enzyme to alterations in pH. Such excipients include but are not limited to epsilon amino caproic acid, lysine, arginine, albumin, human serum albumin, ammonium carbonate and others as taught by Jensen (U.S. Pat. No. 3,950,513).

An interesting addition to the formulations given above is the use of dense formulations to afford “targeting” to the retina post injection. For example, with the patient on their back, injection of the serine proteinase enzyme of interest in any of the formulations given above would be followed by “sinking” of the solution injected towards the retina if that solution were significantly denser than the surrounding vitreous fluid. Agents which can facilitate such density increases include soluble x-ray contrast agents (e.g., Iohexol, Iodixanol, Iomeprol, Ioversol, etc.) concentrated sugar solutions (e.g., sucrose), and heavy metal complexes known to be safe for injection in man (e.g., MRI contrast agents). These agents are able to bring elevated density to formulation for injection and the disclosure herein is not limited to their use but includes all such density adding materials.

A further interesting addition to the formulations given above is the use of viscous formulations to afford delayed diffusion to the retina post injection. For example, with the patient on their back, injection of the serine proteinase enzyme of interest in any of the formulations given above would be followed by delayed diffusion of the solution injected if that solution were significantly more viscous than the surrounding vitreous fluid. Agents which can facilitate such viscosity increases include soluble x-ray contrast agents (e.g., Iohexol, Iodixanol, Iomeprol, Ioversol, etc.), concentrated sugar solutions (e.g., sucrose), solutuble polymers (e.g., PVP, PVA, PEG, etc.), and polymeric surfactants such as Tetronics and Pluronics. These agents are able to bring elevated viscosity to formulation for injection and the disclosure herein is not limited to their use but includes all such viscosity adding materials.

In the special case of the polymeric surfactants, it is known that high concentrations of these materials can induce a reverse thermal gel effect. Thus, upon injection into the vitreous and transition from room temperature to body temperature (e.g., 37° C.), the formulation would “gel” thereby inhibiting the diffusion of the enzyme within the vitreous even more. Delayed diffusion might be important to ensure that the enzyme stays where it is injected rather than traveling from the injection site (i.e., up the needle track of the injection) before diffusing to the retina and other surfaces within the eye.

As examples of the methods of practice given above, serine proteinase enzymes can be formulated as shown in the tables below:

TABLE 1
serine proteinase enzymes for injection

| | | |
|---|---|---|
| Ingredient | Amount per mL | % composition |

| | | | |
|---|---|---|---|
| serine proteinase enzyme | 2.0 | mg | 0.2 |
| Trehalose | 20 | mg | 2 |
| Acetate (Na) | 2.4 | mg | 0.24 |
| Epsilon Amino caproic acid | 3.0 | mg | 0.3 |
| Lysine | 29.2 | mg | 2.92 |
| Normal Saline | QS to 1 | mL | 94.34 |

TABLE 2

| | | |
|---|---|---|
| Ingredient | Amount per mL | % composition |

| | | | |
|---|---|---|---|
| serine proteinase enzyme | 2.0 | mg | 0.2 |
| Trehalose | 20 | mg | 2 |
| Acetate (Na) | 2.4 | mg | 0.24 |
| Epsilon Amino caproic acid | 3.0 | mg | 0.3 |
| Arginine | 34.8 | mg | 3.48 |
| Normal Saline | QS to 1 | mL | 93.78 |

TABLE 3

| | | |
|---|---|---|
| Ingredient | Amount per mL | % composition |

| | | | |
|---|---|---|---|
| serine proteinase enzyme | 2.0 | mg | 0.2 |
| Lactose | 20 | mg | 2 |
| Acetate (Na) | 2.4 | mg | 0.24 |
| Epsilon amino caproic acid | 3.0 | mg | 0.3 |
| Lysine | 29.2 | mg | 2.92 |
| Water for Injection | QS to 1 | mL | 94.34 |

TABLE 4

| | | |
|---|---|---|
| Ingredient | Amount per mL | % composition |

| | | | |
|---|---|---|---|
| serine proteinase enzyme | 2.0 | mg | 0.2 |
| Trehalose | 20 | mg | 2 |
| Citrate (Na) | 4.8 | mg | 0.48 |
| Epslon amino caproic acid | 3.0 | mg | 0.3 |
| Lysine | 29.2 | mg | 2.92 |
| Water for Injection | QS to 1 | mL | 94.10 |

TABLE 5

| | | |
|---|---|---|
| Ingredient | Amount per mL | % composition |

| | | | |
|---|---|---|---|
| serine proteinase enzyme | 2.0 | mg | 0.2 |
| Sucrose | 20 | mg | 2 |
| Acetate (Na) | 2.4 | mg | 0.24 |
| Epsilon amino caproic acid | 3.0 | mg | 0.3 |
| Lysine | 29.2 | mg | 2.92 |
| Normal Saline | QS to 1 | mL | 94.34 |

TABLE 6

| | | |
|---|---|---|
| Ingredient | Amount per mL | % composition |

| | | | |
|---|---|---|---|
| serine proteinase enzyme | 2.0 | mg | 0.2 |
| Sucrose | 20 | mg | 2 |
| Acetate (Na) | 2.4 | mg | 0.24 |
| Epsilon amino caproic acid | 3.0 | mg | 0.3 |
| Arginine | 34.8 | mg | 3.48 |
| Normal Saline | QS to 1 | mL | 93.78 |

TABLE 7

| | | |
|---|---|---|
| Ingredient | Amount per mL | % composition |

| | | | |
|---|---|---|---|
| serine proteinase enzyme | 2.0 | mg | 0.2 |
| Mannitol | 20 | mg | 2 |
| Acetate (Na) | 2.4 | mg | 0.24 |
| Epsilon amino caproic acid | 3.0 | mg | 0.3 |
| Lysine | 29.2 | mg | 2.92 |
| Water for Injection | QS to 1 | mL | 94.34 |

TABLE 8

| | | |
|---|---|---|
| Ingredient | Amount per mL | % composition |

| | | | |
|---|---|---|---|
| serine proteinase enzyme | 2.0 | mg | 0.2 |
| Mannitol | 20 | mg | 2 |
| Acetate (Na) | 2.4 | mg | 0.24 |
| Epsilon amino caproic acid | 3.0 | mg | 0.3 |
| Arginine | 34.8 | mg | 3.48 |
| Normal Saline | QS to 1 | mL | 93.78 |

TABLE 9

| | | |
|---|---|---|
| Ingredient | Amount per mL | % composition |

| | | | |
|---|---|---|---|
| serine proteinase enzyme | 2.0 | mg | 0.2 |
| Trehalose | 20 | mg | 2 |
| Acetate (Na) | 2.4 | mg | 0.24 |
| Epsilon amino caproic acid | 3.0 | mg | 0.3 |
| Human Serum Albumin | 20 | mg | 2.0 |
| Normal Saline | QS to 1 | mL | 95.26 |

TABLE 10

| | | |
|---|---|---|
| Ingredient | Amount per mL | % composition |

| | | | |
|---|---|---|---|
| serine proteinase enzyme | 2.0 | mg | 0.2 |
| Sucrose | 20 | mg | 2 |
| Acetate (Na) | 2.4 | mg | 0.24 |
| Epsilon amino caproic acid | 3.0 | mg | 0.3 |
| Human Serum Albumin | 20 | mg | 2.0 |
| Water for Injection | QS to 1 | mL | 95.26 |

TABLE 11

| Ingredient | Amount per mL | | % composition |
|---|---|---|---|
| serine proteinase enzyme | 2.0 | mg | 0.2 |
| Mannitol | 20 | mg | 2 |
| Acetate (Na) | 2.4 | mg | 0.24 |
| Epsilon amino caproic acid | 3.0 | mg | 0.3 |
| Human Serum Albumin | 29.2 | mg | 2.92 |
| Normal Saline | QS to 1 | mL | 94.34 |

TABLE 12

| Ingredient | Amount per mL | | % composition |
|---|---|---|---|
| serine proteinase enzyme | 2.0 | mg | 0.2 |
| Mannitol | 20 | mg | 2 |
| Citrate (Na) | 4.8 | mg | 0.48 |
| Epsilon amino caproic acid | 3.0 | mg | 0.3 |
| Human Serum Albumin | 29.2 | mg | 2.92 |
| Normal Saline | QS to 1 | mL | 94.10 |

TABLE 13

| Ingredient | Amount per mL | | % composition |
|---|---|---|---|
| serine proteinase enzyme | 2.0 | mg | 0.2 |
| Mannitol | 20 | mg | 2 |
| Acetate (Na) | 2.4 | mg | 0.24 |
| Epsilon amino caproic acid | 3.0 | mg | 0.3 |
| Ammonium Bicarbonate | 10 | mg | 1 |
| Normal Saline | QS to 1 | mL | 96.26 |

TABLE 14

| Ingredient | Amount per mL | | % composition |
|---|---|---|---|
| serine proteinase enzyme | 2.0 | mg | 0.2 |
| Mannitol | 20 | mg | 2 |
| Epsilon amino caproic acid | 3.0 | mg | 0.3 |
| Ammonium Bicarbonate | 10 | mg | 1 |
| Normal Saline | QS to 1 | mL | 96.50 |

TABLE 15

| Ingredient | Amount per mL | | % composition |
|---|---|---|---|
| serine proteinase enzyme | 2.0 | mg | 0.2 |
| Mannitol | 20 | mg | 2 |
| Ammonium Bicarbonate | 15 | mg | 1.5 |
| Epsilon amino caproic acid | 3.0 | mg | 0.3 |
| Human Serum Albumin | 29.2 | mg | 2.92 |
| Normal Saline | QS to 1 | mL | 93.08 |

Tables 1 through 15 detail acceptable formulations for the practice of the method described above. Additionally, formulations using solid tablets can also be used to practice the invention and are represented by the following tables.

TABLE 16

| Ingredient | Amount per Tablet | | % composition |
|---|---|---|---|
| serine proteinase enzyme | 0.05 | mg | 3 |
| Mannitol | 1 | mg | 60.6 |
| Sodium monobasic phosphate | 200 | μg | 12 |
| Sodium dibasic phosphate | 100 | μg | 6 |
| Human Serum Albumin | 100 | μg | 6 |
| Arginine HCl | 200 | μg | 12 |

TABLE 17

| Ingredient | Amount per Tablet | | % composition |
|---|---|---|---|
| serine proteinase enzyme | 0.100 | mg | 6 |
| Dextrose | 1 | mg | 60 |
| Sodium monobasic phosphate | 200 | μg | 12 |
| Sodium dibasic phosphate | 100 | μg | 6 |
| Human Serum Albumin | 100 | μg | 6 |
| Arginine HCl | 200 | μg | 12 |

TABLE 18

| Ingredient | Amount per Tablet | | % composition |
|---|---|---|---|
| serine proteinase enzyme | 0.05 | mg | 3 |
| Sucrose | 1 | mg | 60.6 |
| Sodium monobasic phosphate | 200 | μg | 12 |
| Sodium dibasic phosphate | 100 | μg | 6 |
| Human Serum Albumin | 100 | μg | 6 |
| Arginine HCl | 200 | μg | 12 |

TABLE 19

| Ingredient | Amount per Tablet | | % composition |
|---|---|---|---|
| serine proteinase enzyme | 0.08 | mg | 5 |
| Mannitol | 1 | mg | 60 |
| Sodium monobasic phosphate | 200 | μg | 12 |
| Sodium dibasic phosphate | 100 | μg | 6 |
| Human Serum Albumin | 100 | μg | 6 |
| Lysine | 200 | μg | 12 |

TABLE 20

| Ingredient | Amount per Tablet | | % composition |
|---|---|---|---|
| serine proteinase enzyme | 0.100 | mg | 6 |
| Dextrose | 1 | mg | 60 |
| Sodium monobasic phosphate | 200 | μg | 12 |
| Sodium dibasic phosphate | 100 | μg | 6 |
| Human Serum Albumin | 100 | μg | 6 |
| Lysine | 200 | | 12 |

The tablets represented above in Tables 16 through 20 are rapidly dissolving tablets which release the active enzyme within 30 min post dosing into the vitreous of the eye.

### EXAMPLES

The following examples illustrate how the invention may be used for non-surgically preventing or reducing the rate of the progression of non-proliferative diabetic retinopathy and for treating other ocular conditions.

### Example 1

A formulation containing 5% saccharide (e.g., trehalose, manose, dextrose, fructose, xylose, galactose) with a small amount of buffer (e.g., acetate, citrate), an amount equivalent to about 2.0 mg per ml of plasmin (which amount varies depending on eye volume), and optionally containing a plasmin stabilizer (e.g., a dibasic amino acid or derivative thereof such as epsilon amino caproic acid) at a 3.0<pH<8.0 is injected into the vitreous through the pars plana, using a 27 ga needle, of a patient suffering from non-proliferative diabetic retinopathy. The concentration of plasmin is sufficient to create a posterior vitreal detachment (PVD) with one injection without surgery. The PVD is confirmed by conventional ocular exam, optical coherence tomography, beta scan ultrasound alone or in any combination thereof. If a PVD cannot be confirmed, one or more subsequent injections may be made. The creation of the PVD prevents or reduces the risk of the progression of non-proliferative diabetic retinopathy to the proliferative form of diabetic retinopathy.

### Example 2

A formulation containing 5% saccharide (e.g., trehalose, manose, dextrose, fructose, xylose, galactose) with a small amount of buffer (e.g., acetate, citrate), an amount equivalent to about 2.0 mg per ml of plasmin (which amount varies depending on eye volume), and optionally containing a plasmin stabilizer (e.g., a dibasic amino acid or derivative thereof such as epsilon amino caproic acid) at a 3.0<pH<8.0 is injected into the vitreous through the pars plana, using 27 ga needle, prior to cataract surgery, to induce a PVD as a prophylaxis against post surgical macular edema in diabetic patients. The prophylactic procedure would be applicable to diabetic patients exhibiting clinically significant macular edema prior to surgery or to diabetic patients in general due to undergo cataract surgery. The concentration of plasmin is sufficient to create a posterior vitreal detachment (PVD) with one injection without surgery. The PVD is confirmed by conventional ocular exam, optical coherence tomography, beta scan ultrasound alone or in any combination thereof. If a PVD cannot be confirmed, one or more subsequent injections may be made. The creation of the PVD prevents or reduces the risk of post surgical macular edema in patients undergoing cataract surgery.

The injection of plasmin to induce prophylactic PVD prior to cataract surgery would also apply to patients with high myopia requiring cataract surgery, clear lens exchange or any other intra-ocular refractive procedure.

### Example 3

Following diagnosis of a patient at risk of retinal detachment (e.g. the presence of a clinically significant vitreoretinal membrane and traction or presence of a vitreoretinal degenerative disorder and retinal detachment has occurred already in the other eye), an intravitreal injection of plasmin is made, using the formulation and procedure described in Example 1, into the vitreous at a dose sufficient to enzymatically cleave the vitreoretinal membrane and cause disinsertion of the vitreous respectively without surgery, thereby preventing retinal detachment.

### Example 4

Following diagnosis of vitreoretinal traction causing maculopathy or retinopathy, an intravitreal injection of plasmin is made, using the formulation and procedure described in Example 1, into the posterior vitreous at a dose sufficient to cause disinsertion of the posterior vitreous, without surgery, thereby treating tractional maculopathy or tractional retinopathy.

### Example 5

A formulation as in example 1 wherein the formulation is lyophilized under conditions known in the art to provide a stable solid cake that can be reconstituted with water for injection, normal saline, or phosphate buffered saline to provide a clear solution for injection into the vitreous. Reconstitution volumes depend upon the final concentration required to treat the diseases disclosed herein and on the size of the eye to be treated; however, it is preferred that for a cake containing 25 mg of plasmin, enough solvent is added to reconstitute to 5 mg/ml in plasmin concentration and even more preferred to add enough solvent to make the resulting solution 2 mg/ml plasmin. While many different solvents can be used to reconstitute the lyophilized cake, water for injection, normal saline and phosphate buffered saline are preferred. Even more preferred are water for injection and normal saline and most preferred is normal saline. It is equally clear that the presence of a stabilizer for plasmin can be in the freeze dried cake or in the solvent for protection of the plasmin from pH shifts upon injection into the vitreous.

### Example 6

Following intravitreal injection of a chemical spreading agent, e.g. Vitrase® or hylauronidase, an intravitreal injection of plasmin is made, using the formulation and procedure described in Example 1, into the posterior vitreous at a dose sufficient to cause disinsertion of the posterior vitreous, without surgery, thereby treating tractional maculopathy or tractional retinopathy.

Although the invention has been described in connection with various preferred embodiments, numerous variations will be apparent to a person of ordinary skill in the art given the present description, without departing from the spirit of the invention and the scope of the appended claims. For example, modifications to the preferred embodiments will be evident when the invention is used for different ocular conditions or when the invention is used in different formulations.

We claim:

1. A stabilized, ophthalmic plasmin formulation upon reconstitution comprising:
a) about 0.10 to about 10 mg per ml of plasmin;
b) about 0.10 mg to about 100 mg per ml of a saccharide or saccharide derivative;
c) a buffer;
d) a pH of from about 2.0 to about 5.0; and,
e) and effective amount of a plasmin stabilizer to prevent rapid shifts in plasmin physical stability during pH changes in the ophthalmic plasmin formulation or upon injection into the vitreous.

2. A stabilized, ophthalmic plasmin formulation according to claim 1, wherein said plasmin is about 0.02 to about 10 mg per ml of plasmin.

3. A stabilized, ophthalmic plasmin formulation according to claim 1, wherein said plasmin is about 0.03 to about 5 mg per ml of plasmin.

4. A stabilized, ophthalmic plasmin formulation according to claim 1, wherein said plasmin is about 0.03 to about 4 mg per ml of plasmin.

5. A stabilized, ophthalmic plasmin formulation according to claim 1, wherein said plasmin is about 2.0 mg per ml of plasmin.

6. A stabilized, ophthalmic plasmin formulation according to claim 1, wherein said saccharide is selected from the group comprising trehalose, lactose, sucrose, manose, dextrose, fructose, xylose, galactose or a saccharide derivative.

7. A stabilized, ophthalmic plasmin formulation according to claim 1, wherein said saccharide is trehalose.

8. A stabilized ophthalmic plasmin formulation according to claim 1, wherein said saccharide derivative is selected from the group comprising mannitol, sortitol or xylitol.

9. A stabilized ophthalmic plasmin formulation according to claim 1, wherein said buffer is selected from the group comprising acetate, citrate and phosphate.

10. A stabilized ophthalmic plasmin formulation according to claim 1, wherein said buffer is acetate.

11. A stabilized ophthalmic plasmin formulation according to claim 1, wherein said pH is about 3 to 5.

12. A stabilized ophthalmic plasmin formulation according to claim 1, wherein said pH is about 3.5 to 5.

13. A stabilized ophthalmic plasmin formulation according to claim 1, wherein said pH is about 3 to 4.

14. A stabilized ophthalmic plasmin formulation according to claim 1, wherein said effective amount of a plasmin stabilizer is about 1 mM to about 100 mM.

15. A stabilized ophthalmic plasmin formulation according to claims 1 and 66, wherein said plasmin stabilizer is a dibasic amino acid or derivative thereof.

16. A stabilized ophthalmic plasmin formulation according to claims 1 and 66, wherein said plasmin stabilizer is selected from the group comprising epsilon amino caproic acid, lysine, arginine, glycylglycine.

17. A stabilized ophthalmic plasmin formulation according to claims 1 and 66, wherein said plasmin stabilizer is epsilon amino caproic acid.

18. A stabilized, ophthalmic plasmin formulation comprising:
a. about 0.01 to about 10 mg per ml of plasmin;
b. about 10 mg to about 50 mg of trehalose;
c. an acetate or citrate buffer;
d. a pH from about 3 to about 5; and
e. about 1 mM to about 100 mM of a dibasic amino acid or derivative thereof to prevent rapid shifts in plasmin physical stability during pH changes in the ophthalmic plasmin formulation or upon injection into the vitreous.

19. A stabilized, ophthalmic plasmin formulation comprising:
f. about 2.0 mg per ml of plasmin;
g. about 20 mg of trehalose;
h. an acetate buffer,
i. a pH from about 3 to 5; and
j. from about 3 mM of epsilon amino caproic acid to prevent rapid shifts in plasmin physical stability during pH changes in the ophthalmic plasmin formulation or upon injection into the vitreous.

20. A method according to any one of claim 1, 18 or 19, wherein a viscosity enhancer is added to said formulation.
